SEQUENCE LISTING (5 sequences)

SEQ ID NO: 1 - RNA, 22 nt, Homo sapiens
caguuaucacagugcugaugcu

SEQ ID NO: 2 - RNA, 23 nt, Homo sapiens
guccaguuuucccaggaaucccu

SEQ ID NO: 3 - RNA, 22 nt, Homo sapiens
caucuuccaguacaguguugga

SEQ ID NO: 4 - RNA, 21 nt, Homo sapiens
uagcagcacagaaauauuggc

SEQ ID NO: 5 - RNA, 22 nt, Homo sapiens
uuuguucguucggcucgcguga

(19) United States
(12) Patent Application Publication
(10) Pub. No.: US 2018/0371554 A1
(43) Pub. Date: Dec. 27, 2018

(54) METHODS FOR DIAGNOSING OR PROGNOSING PROSTATE CANCER

(71) Applicants: INSERM (INSTITUT NATIONAL DE LA SANTÉ ET DE LA RECHERCHE MÉDICALE), Paris (FR); UNIVERSITE NICE SOPHIA ANTIPOLIS, Nice (FR)

(72) Inventor: Mohamed BENAHMED, Nice Cedex 3 (FR)

(21) Appl. No.: 16/062,334

(22) PCT Filed: Dec. 14, 2016
(86) PCT No.: PCT/EP2016/080992

§ 371 Date: Jun. 14, 2018

(30) Foreign Application Priority Data
Dec. 15, 2015 (EP) 15307005.7

(51) Int. Cl. C12Q 1/6886

(52) U.S. Cl. CPC C12Q 1/6886; C12Q 2600/112; C12Q 2600/178; C12Q 2600/158; C12Q 2600/118

(57) ABSTRACT

The present invention relates to methods and kits for diagnosing or prognosing prostate cancer. More particularly, the invention relates to an in vitro method for predicting or diagnosing a prostate cancer in a subject, said method comprising the following steps of: i) measuring the expression levels of miR-101 and miR-145 in a biological sample obtained from said subject; ii) comparing said expression levels of miR-101 and miR-145 with their respective predetermined reference values; and iii) determining whether the subject has or is at risk of having a prostate cancer, wherein an expression level of miR-101 lower than the predetermined reference value and an expression level of miR-145 higher than the predetermined reference value is indicative that the subject has, or is at risk of having a prostate cancer.

### FIELD OF THE INVENTION

The present invention relates generally to the field of oncology. More specifically, the present invention relates to methods and kits for diagnosing or prognosing prostate cancer.

### BACKGROUND OF THE INVENTION

The introduction of prostate-specific antigen (PSA) screening in 1987 has led to the diagnosis and aggressive treatment of many cases of indolent prostate cancer that would never have become clinically significant or caused death. The reason for this is that the natural history of prostate cancer is unusual among malignancies in that the majority of cases are indolent and even if untreated would not progress during the course of a man's life to cause suffering or death. While approximately half of men develop invasive prostate cancer during their lifetimes, only 20% will be diagnosed with prostate cancer and only 3% will die as a result of prostate cancer. However, currently, over 90% of men who are diagnosed with prostate cancer, even low-risk prostate cancer, are treated with either immediate radical prostatectomy or definitive radiation therapy. This over-treatment of prostate cancer comes at a cost of money and toxicity. For example, the majority of men who undergo radical prostatectomy suffer incontinence and impotence as a result of the procedure, and as many as 25% of me regret their choice of treatment for prostate cancer.

One of the reasons for the over-treatment of prostate cancer is the lack of adequate prognostic tools to distinguish men who need immediate definitive therapy from those who are appropriate candidates to defer immediate therapy and undergo active surveillance instead. For example, of men who appear to have low-risk disease based on the results of clinical staging, pre-treatment PSA, and biopsy Gleason score, and have been managed with active surveillance on protocols. 30-40% experience disease progression (diagnosed by rising PSA, an increased Gleason score on repeat biopsy, or clinical progression) over the first, few years of follow-up, and some of them may have lost the opportunity for curative therapy. Also, of men who appear to be candidates for active surveillance, hut who undergo immediate prostatectomy anyway, 30-40% are found at surgery to have higher risk disease than expected as defined by having high-grade (Gleason score of 3+4 or higher) or non-organ-confined disease (extracapsular extension (ECE) or seminal vesicle involvement (SVI)).

Estimates of recurrence risk and treatment decisions in prostate cancer are currently based primarily on PSA levels and/or clinical tumor stage. Although clinical tumor stage has been demonstrated to have a significant association with outcome, sufficient to be included in pathology reports, variations in approach to the acquisition, interpretation, reporting, and analysis of this information exist. As a consequence, existing pathologic staging methods have been criticized as lacking reproducibility and therefore may provide imprecise estimates of individual patient risk. Indeed, it has been recently realized that pre-treatment PSA levels, the primary predictive parameter in the majority of tools to predict recurrence, may reflect primarily the presence of benign prostatic hyperplasia (BPH) rather than prostate cancer. PSA is thus not specific for this malignancy, being elevated in many other conditions BPH. Perhaps more important than its diagnostic inaccuracy, three large clinical trials have revealed that PSA testing/screening is associated with a high rate of overdiagnosis and overtreatment.

It results that, early identification of men with localized prostate cancer as well as of patients newly diagnosed likely to ultimately experience recurrence would be useful in considering additional treatments while preserving quality of life. Further, increased accuracy in the classification of newly diagnosed clinically localized prostate cancers is needed if treatment is to be better tailored to this subgroup of patients. And also accurate estimation of a likelihood of recurrence will also be useful in clinical trials to identify candidates for control groups or for an investigational treatment of interest.

While a number of molecules other than PSA are associated with prostate cancer, it is unclear whether any of these molecules, or combinations thereof, are useful to predict disease outcome. Therefore, there is an imminent need for novel markers that are specifically associated with prostate cancer for early diagnosis as well as improved prediction of outcome in patients newly diagnosed with a clinically localized prostate cancer. Amongst these molecules, emerging evidence suggests that circulating or urine miRNAs are useful as noninvasive biomarkers of prostate cancer as described in Sapre & Selth 2013.

### SUMMARY OF THE INVENTION

The present invention relates to methods and kits for predicting or diagnosing a prostate cancer in a subject by determining the level of miR in a biological sample. In particular, the invention is defined by the claims.

### DETAILED DESCRIPTION OF THE INVENTION

The present invention is based on the discovery that a particular combination of miRs (i.e. at least miR-101 and miR-145, with preferably miR-141 and/or miR-195 and/or miR-375) allow to predict with an optimal sensitivity (at least superior to 90%, preferably superior to 95% and even more preferably equal to 100% comparatively with the 70% obtained by measuring PSA), the organ localized prostate disease status, the potential for progression of prostate cancer in patients notably following primary therapy, and the likelihood of a recurrence of prostate cancer. The combination of the invention is thus particularly useful for diagnosing subjects with localized prostate cancers as well as for evaluating patients at risk for recurrence of prostate cancer in diverse clinical situations for patients including pre-prostatectomy, post-prostatectomy, pre-radiation therapy and post-radiation therapy. In addition to improving diagnosis and prognosis, knowledge of the disease status allows the attending physician to select the most appropriate therapy for the individual patient.

### Definitions

Throughout the specification, several terms are employed and are defined in the following paragraphs.

The term “cancer” refers to or describes the physiological condition in mammals that is typically characterized by unregulated cell growth.

As used herein, the term “prostate cancer” is used in the broadest sense and refers to all stages and all forms of cancer arising from the tissue of the prostate gland. The term “prostate cancer” encompasses any type of malignant (i.e. non benign) tumor located in prostatic tissues, such as e.g. prostatic adenocarcinoma, prostatic sarcoma, undifferentiated prostate cancer, prostatic squamous cell carcinoma, prostatic ductal transitional carcinoma and prostatic intraepithelial neoplasia.

Staging of the cancer assists a physician in assessing how far the disease has progressed and to plan a treatment for the patient. Staging may be done clinically (clinical staging) by physical examination, blood tests, or response to radiation therapy, and/or pathologically (pathologic staging) based on surgery, such as radical prostatectomy.

According to the tumor, node, metastasis (TNM) staging system of the American Joint Committee on Cancer (AJCC), AJCC Cancer Staging Manual (7th Ed., 2010), the various stages of prostate cancer are defined as follows: Tumor: T1: clinically inapparent tumor not palpable or visible by imaging, T1a: tumor incidental histological finding in 5% or less of tissue resected, T1b: tumor incidental histological finding in more than 5% of tissue resected, Tic: tumor identified by needle biopsy; T2: tumor confined within prostate, T2a: tumor involves one half of one lobe or less, T2b: tumor involves more than half of one lobe, but not both lobes, T2c: tumor involves both lobes; T3: tumor extends through the prostatic capsule, T3a: extracapsular extension (unilateral or bilateral), T3b: tumor invades seminal vesicle(s); T4: tumor is fixed or invades adjacent structures other than seminal vesicles (bladder neck, external sphincter, rectum, levator muscles, or pelvic wail). Generally, a clinical T (cT) stage is T1 or T2 and pathologic T (pT) stage is T2 or higher. Node: N0: no regional lymph node metastasis; N1: metastasis in regional lymph nodes. Metastasis: M0: no distant metastasis; M1: distant metastasis present.

The Gleason Grading system is used to help evaluate the prognosis of men with prostate cancer. Together with other parameters, it is incorporated into a strategy of prostate cancer staging, which predicts prognosis and helps guide therapy. A Gleason “score” or “grade” is given to prostate cancer based upon its microscopic appearance. Tumors with a low Gleason score typically grow slowly enough that they may not pose a significant threat to the patients in their lifetimes. These patients are monitored (“watchful waiting” or “active surveillance”) over time. Cancers with a higher Gleason score are more aggressive and have a worse prognosis, and these patients are generally treated with surgery (e.g., radical prostatectomy) and, in some cases, therapy (e.g., radiation, hormone, ultrasound, chemotherapy). Gleason scores (or sums) comprise grades of the two most common tumor patterns. These patterns are referred to as Gleason patterns 1-5, with pattern 1 being the most well-differentiated. Most have a mixture of patterns. To obtain a Gleason score or grade, the dominant pattern is added to the second most prevalent pattern to obtain a number between 2 and 10. The Gleason Grades include: G1: well differentiated (slight anaplasia) (Gleason 2-4); G2: moderately differentiated (moderate anaplasia) (Gleason 5-6); G3-4: poorly differentiated/undifferentiated (marked anaplasia) (Gleason 7-10).

Stage groupings: Stage I; T1a N0 M0 G1; Stage II: (T1a N0 M0 G2-4) or (T1b, c, T1, T2, N0 M0 Any G); Stage III: T3 N0 M0 Any G; Stage IV: (T4 N0 M0 Any G) or (Any T1 M0 Any G) or (Any T Any N M1 Any G).

The terms “organ-confined” or “localized” as used herein refer to pathologic stage pT2 at RP. The term “non organ-confined disease” as used herein refers to having pathologic stage T3 disease at RP.

The term “miRNAs” (also called “miR”) has its general meaning in the art and refers to microRNA molecules that are generally 21 to 22 nucleotides in length, even though lengths of 19 and up to 23 nucleotides have been reported. miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem. The processed miRNA (also referred to as “mature miRNA”) become part of a large complex to down-regulate a particular target gene.

All the miRNAs pertaining to the invention are known per se and sequences of them are publicly available from the data base http://microrna.sanger.ac.uk/sequences/.

The miRNAs of the invention are listed in Table A:

TABLE A
list of the miRNAs according to the invention

| miRNA (Sequence ID) | miRBase Accession number |
|---|---|
| (miR-101) (SEQ ID NO: 1) | MI0000103 |
| caguuaucacagugcugaugcu | |
| (miR-145) (SEQ ID NO: 2) | MI0000461 |
| guccaguuuucccaggaaucccu | |
| (miR-141) (SEQ ID NO: 3) | MI0000457 |
| caucuuccaguacaguguugga | |
| (miR-195) (SEQ ID NO: 4) | MI0000489 |
| uagcagcacagaaauauuggc | |
| (miR-375) (SEQ ID NO: 5) | MI0000783 |
| uuuguucguucggcucgcguga | |

As used herein, the term “measuring” encompasses detecting or quantifying.

As used herein, “detecting” means determining if a miR (e.g. miR-101) is present or not in a biological sample and “quantifying” means determining the amount of a miR (e.g. miR-101) in a biological sample.

### Diagnostic Methods

In a first aspect, the present invention relates to an in vitro method for predicting or diagnosing a prostate cancer in a subject, said method comprising the following steps of:
i. measuring the expression levels of miR-101 and miR-145 in a biological sample obtained from said subject;
ii. comparing said expression levels of miR-101 and miR-145 with their respective predetermined reference values; and
iii. determining whether the subject has or is at risk of having a prostate cancer, wherein an expression level of miR-101 lower than the predetermined reference value and an expression level of miR-145 higher than the predetermined reference value is indicative that the subject has, or is at risk of having a prostate cancer.

In a particular embodiment, the method further comprises a step of measuring the expression level of miR-141.

Thus, the method comprises the following steps of:
i. measuring the expression levels of miR-101, miR-145 and miR-141 in a biological sample obtained from said subject;
ii. comparing said expression levels of miR-101, miR-145 and miR-141 with their respective predetermined reference values; and
iii. determining whether the subject has or is at risk of having a prostate cancer, wherein an expression level of miR-101 lower than the predetermined reference value, an expression level of miR-145 higher than the predetermined reference value and an expression level of miR-141 higher than the predetermined reference value is indicative that the subject has, or is at risk of having a prostate cancer.

In another particular embodiment, the method further comprises a step of measuring the expression level of miR-195.

Thus, the method comprises the following steps of:
i. measuring the expression levels of miR-101, miR-145 and miR-195 in a biological sample obtained from said subject;
ii. comparing said expression levels of miR-101, miR-145 and miR-195 with their respective predetermined reference values; and
iii. determining whether the subject has or is at risk of having a prostate cancer, wherein an expression level of miR-101 lower than the predetermined reference value, an expression level of miR-145 higher than the predetermined reference value and an expression level of miR-195 higher than the predetermined reference value is indicative that the subject has, or is at risk of having a prostate cancer.

In a preferred embodiment, the method further comprises a step of measuring the expression levels of miR-141 and miR-195.

Thus, the method comprises the following steps of:
i. measuring the expression levels of miR-101, miR-145, miR-141 and miR-195 in a biological sample obtained from said subject;
ii. comparing said expression levels of miR-101, miR-145, miR-141 and miR-195 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-141 higher than the predetermined reference value and an expression level of miR-195 higher than the predetermined reference value is indicative that the subject has, or is at risk of having a prostate cancer.

In another particular embodiment, the method further comprises a step of measuring the expression level of miR-375.

Thus, the method comprises the following steps of:
i. measuring the expression levels of miR-101, miR-145 and miR-375 in a biological sample obtained from said subject;
ii. comparing said expression levels of miR-101, miR-145 and miR-375 with their respective predetermined reference values; and
iii. determining whether the subject has or is at risk of having a prostate cancer, wherein an expression level of miR-101 lower than the predetermined reference value, an expression level of miR-145 higher than the predetermined reference value and an expression level of miR-375 higher than the predetermined reference value is indicative that the subject has, or is at risk of having a prostate cancer.

In a preferred embodiment, the method further comprises a step of measuring the expression levels of miR-375 and miR-141.

Thus, the method comprises the following steps of:
i. measuring the expression levels of miR-101, miR-145, miR-141 and miR-375 in a biological sample obtained from said subject;
ii. comparing said expression levels of miR-101, miR-145, miR-141 and miR-375 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-141 higher than the predetermined reference value and an expression level of miR-375 higher than the predetermined reference value is indicative that the subject has, or is at risk of having a prostate cancer.

In another preferred embodiment, the method further comprises a step of measuring the expression levels of miR-375 and miR-195.

Thus, the method comprises the following steps of:
i. measuring the expression levels of miR-101, miR-145, miR-195 and miR-375 in a biological sample obtained from said subject;
ii. comparing said expression levels of miR-101, miR-145, miR-195 and miR-375 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-195 higher than the predetermined reference value and an expression level of miR-375 higher than the predetermined reference value is indicative that the subject has, or is at risk of having a prostate cancer.

In still another preferred embodiment, the method further comprises a step of measuring the expression levels of miR-375, miR-141 and miR-195.

Thus, the method comprises the following steps of:
i. measuring the expression levels of miR-101, miR-145, miR-141, miR-195 and miR-375 in a biological sample obtained from said subject;
ii. comparing said expression levels of miR-101, miR-145, miR-141, miR-195 and miR-375 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-141 higher than the predetermined reference value, an expression level of miR-195 higher than the predetermined reference value and an expression level of miR-375 higher than the predetermined reference value is indicative that the subject has, or is at risk of having a prostate cancer.

At step ii), the comparison step may be obtained by comparing the expression level in the biological sample from the subject with expression level in a biological sample from a healthy subject (or group of healthy subjects). A higher expression is indicative of that the subject has, or is at risk of having a prostate cancer.

Thus, as used herein, a “higher expression level” consists of a an expression level value that is statistically (i.e. significantly) higher than the predetermined reference value (that may also be termed the “control” expression value or “control reference” values) that has been previously determined in the same biological sample from a healthy subject, e.g. a blood sample from a healthy subject. Alternatively, the control may also be obtained by measuring expression level of miRs in the normal tissue adjacent to the tumor of the same cancer patient.

In a particular embodiment, the predetermined reference value is a threshold value or a cut-off value that can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the expression levels (obtained according to the method of the invention) with a defined threshold value.

Any subject sample suspected of containing miRs may be tested according to the methods of the invention. By way of non-limiting examples, the biological sample may be tissue (e.g., a prostate biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

Accordingly, measuring the expression level of a miR such as miR-101 and miR-145 in a biological sample obtained from the subject may be performed by a variety of techniques.

For example the nucleic acid contained in the biological sample (e.g., a biopsy sample or a blood sample prepared from the subject) is first extracted according to standard methods, for example using lytic enzymes or chemical solutions or extracted by nucleic-acid-binding resins following the manufacturer's instructions. The extracted miRNAs is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence based amplification (NASBA).

In a particular embodiment, the determination comprises contacting the sample with selective reagents such as probes or primers and thereby detecting the presence, or measuring the amount of miRNAs originally in the sample. Contacting may be performed in any suitable device, such as a plate, microtiter dish, test tube, well, glass, column, and so forth. In specific embodiments, the contacting is performed on a substrate coated with the reagent, such as a miRNA array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a miRNAs hybrid, to be formed between the reagent and the miRNAs of the sample.

Nucleic acids exhibiting sequence complementarity or homology to the miRNAs of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin biotin).

The probes and primers are “specific” to the miRNAs they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

Accordingly, the invention concerns the preparation and use of miRNA arrays or miRNA probe arrays, which are macroarrays or microarrays of nucleic acid molecules (probes) that are fully or nearly complementary or identical to a plurality of miRNA molecules positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like.

After an array or a set of miRNA probes is prepared and/or the miRNA in the sample or miRNA probe is labelled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001). Of particular interest in many embodiments is the use of stringent conditions during hybridization. Stringent conditions are known to those of skill in the art.

Alternatively, miRNAs quantification method may be performed by using stem-loop primers for reverse transcription (RT) followed by a real-time TaqMan® probe. Typically, said method comprises a first step wherein the stem-loop primers are annealed to miRNA targets and extended in the presence of reverse transcriptase. Then miRNA-specific forward primer, TaqMan® probe, and reverse primer are used for PCR reactions. Quantitation of miRNAs is estimated based on measured CT values.

Many miRNA quantification assays are commercially available from Qiagen (S. A. Courtaboeuf, France) or Applied Biosystems (Foster City, USA).

Expression level of a miRNA may be expressed as absolute expression level or normalized expression level. Typically, expression levels are normalized by correcting the absolute expression level of a miRNA by comparing its expression to the expression of a mRNA that is not a relevant for determining subject having or at risk of having or developing an infertility, e.g., a housekeeping RNA that is constitutively expressed. Suitable RNA for normalization includes housekeeping RNAs such as the U6, U24, U48, S18 and cel-miR-39. This normalization allows the comparison of the expression level in one sample, e.g., a subject sample, to another sample, or between samples from different sources.

### Prognostic Methods

In a second aspect, the present invention relates to an in vitro method for predicting the clinical outcome for a patient diagnosed with prostate cancer, said method comprising the following steps of:
i. measuring the expression levels of miR-101 and miR-145 in a biological sample obtained from said subject;
ii. comparing said expression levels of miR-101 and miR-145 with their respective predetermined reference values; and
iii. predicting the clinical outcome for said patient, wherein an expression level of miR-101 lower than the respective predetermined reference value and an expression level of miR-145 higher than the respective predetermined reference value is indicative that the patient has a decreased likelihood of a positive clinical outcome.

The term “prediction” is used herein to refer to the likelihood that a patient will have a particular clinical outcome, whether positive or negative, prior to or after primary therapy.

The term “positive clinical outcome” means an improvement in any measure of patient status, including those measures ordinarily used in the art. A “positive clinical outcome” may be assessed using any endpoint indicating a benefit to the patient, including, without limitation, (1) inhibition, to some extent, of tumor growth, including slowing down and complete growth arrest; (2) reduction in the number of tumor ceils; (3) reduction in tumor size; (4) inhibition (i.e., reduction, slowing down, or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor: (8) increase in the duration of survival following treatment; and/or (9) decreased mortality at a given point of time following treatment. Positive clinical outcome can also be considered in the context of an individual's outcome relative to an outcome of a population of patients having a comparable clinical diagnosis, and can be assessed using various endpoints such as an increase in the duration of clinical Recurrence-Free Interval (cRFI), an increase in survival time (Overall Survival (OS)) or prostate cancer-specific survival time (Prostate Cancer-Specific Survival (PCSS) in a population, no upstaging or upgrading in tumor stage or Gleason grade between biopsy and radical prostatectomy, presence of 3+3 grade and organ-confined disease at radical prostatectomy, and the like.

The term “clinical recurrence-free interval (cRFI)” is used herein as time from surgery to first clinical recurrence or death due to clinical recurrence of prostate cancer.

The term “Overall Survival (OS)” is used herein to refer to the time from surgery to death from any cause. The term “Prostate Cancer-Specific Survival (PCSS)” is used herein to describe the time from surgery to death from prostate cancer.

The term “upgrading” as used herein refers to an increase in Gleason grade determined from biopsy to Gleason grade determined from radical prostatectomy (RP). For example, upgrading includes a change in Gleason grade from 3+3 or 3+4 on biopsy to 3+4 or greater on RP. “Significant upgrading” or “upgrade2” as used herein, refers to a change in Gleason grade from 3+3 or 3+4 determined from biopsy to 4+3 or greater, or seminal vessical involvement (SVI), or extracapsular involvement (ECE) as determined from RP.

The term “high grade” as used herein refers to Gleason score of >=3+4 or >=4+3 on RP. The term “low grade” as used herein refers to a Gleason score of 3+3 on RP.

The term “upstaging” as used herein refers to an increase in tumor stage from biopsy to tumor stage at RP. For example, upstaging is a change in tumor stage from clinical T1 or T2 stage at biopsy to pathologic T3 stage at RP.

In a particular embodiment, the method further comprises a step of measuring the expression level of miR-141.

Thus, the method comprises the following steps of:
i. measuring the expression levels of miR-101, miR-145 and miR-141 in a biological sample obtained from said subject;
ii. comparing said expression levels of miR-101, miR-145 and miR-141 with their respective predetermined reference values; and
iii. determining whether the subject has or is at risk of having a prostate cancer, wherein an expression level of miR-101 lower than the predetermined reference value, an expression level of miR-145 higher than the predetermined reference value and an expression level of miR-141 higher than the predetermined reference value is indicative that the patient has a decreased likelihood of a positive clinical outcome.

In another particular embodiment, the method further comprises a step of measuring the expression level of miR-195.

Thus, the method comprises the following steps of:
i. measuring the expression levels of miR-101, miR-145 and miR-195 in a biological sample obtained from said subject;
ii. comparing said expression levels of miR-101, miR-145 and miR-195 with their respective predetermined reference values; and
iii. determining whether the subject has or is at risk of having a prostate cancer, wherein an expression level of miR-101 lower than the predetermined reference value, an expression level of miR-145 higher than the predetermined reference value and an expression level of miR-195 higher than the predetermined reference value is indicative that the patient has a decreased likelihood of a positive clinical outcome.

In a preferred embodiment, the method further comprises a step of measuring the expression levels of miR-141 and miR-195.

Thus, the method comprises the following steps of:
i. measuring the expression levels of miR-101, miR-145, miR-141 and miR-195 in a biological sample obtained from said subject;
ii. comparing said expression levels of miR-101, miR-145, miR-141 and miR-195 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-141 higher than the predetermined reference value and an expression level of miR-195 higher than the predetermined reference value is indicative that the patient has a decreased likelihood of a positive clinical outcome.

In another particular embodiment, the method further comprises a step of measuring the expression level of miR-375.

Thus, the method comprises the following steps of:
i. measuring the expression levels of miR-101, miR-145 and miR-375 in a biological sample obtained from said subject;
ii. comparing said expression levels of miR-101, miR-145 and miR-375 with their respective predetermined reference values; and
iii. determining whether the subject has or is at risk of having a prostate cancer, wherein an expression level of miR-101 lower than the predetermined reference value, an expression level of miR-145 higher than the predetermined reference value and an expression level of miR-375 higher than the predetermined reference value is indicative that the patient has a decreased likelihood of a positive clinical outcome.

In a preferred embodiment, the method further comprises a step of measuring the expression levels of miR-375 and miR-141.

Thus, the method comprises the following steps of:
i. measuring the expression levels of miR-101, miR-145, miR-375 and miR-141 in a biological sample obtained from said subject;
ii. comparing said expression levels of miR-101, miR-145, miR-375 and miR-141 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-375 higher than the predetermined reference value and an expression level of miR-141 higher than the predetermined reference value is indicative that the patient has a decreased likelihood of a positive clinical outcome.

In another preferred embodiment, the method further comprises a step of measuring the expression levels of miR-375 and miR-195.

Thus, the method comprises the following steps of:
i. measuring the expression levels of miR-101, miR-145, miR-375 and miR-195 in a biological sample obtained from said subject;
ii. comparing said expression levels of miR-101, miR-145, miR-375 and miR-195 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-375 higher than the predetermined reference value and an expression level of miR-195 higher than the predetermined reference value is indicative that the patient has a decreased likelihood of a positive clinical outcome.

In still another preferred embodiment, the method further comprises a step of measuring the expression levels of miR-375, miR-141 and miR-195.

Thus, the method comprises the following steps of:
i. measuring the expression levels of miR-101, miR-145, miR-375, miR-141 and miR-195 in a biological sample obtained from said subject;
ii. comparing said expression levels of miR-101, miR-145, miR-375, R-141 and miR-195 with their respective predetermined reference values; and
iii. determining whether the subject has or is at risk of having a prostate cancer, wherein an expression level of miR-101 lower than the predetermined reference value, an expression level of miR-145 higher than the predetermined reference value, an expression level of miR-375 higher than the predetermined reference value, an expression level of miR-141 higher than the predetermined reference value and an expression level of miR-195 higher than the predetermined reference value is indicative that the patient has a decreased likelihood of a positive clinical outcome.

Measuring the expression level of miRs in a biological sample obtained from the patient may be performed by a variety of techniques as previously described.

In one embodiment, the patient is newly diagnosed with a clinically localized prostate cancer prior to or after primary therapy for clinically localized prostate cancer.

In a particular embodiment, the patient is diagnosed with a prostate cancer with a Gleason score of =3+4 or =4+3.

In another particular embodiment, the patient is under active surveillance.

As used herein, the terms “active surveillance” and “watchful waiting” mean closely monitoring a patient's condition without giving any treatment until symptoms appear or change. For example, in prostate cancer, watchful waiting is usually used in older men with other medical problems and early-stage disease.

In still another particular embodiment, the primary therapy for is surgery, radiation therapy including brachytherapy and external beam radiation therapy, high-intensity focused ultrasound (HIFU), chemotherapy, cryosurgery, hormonal therapy, or combination thereof.

As used herein, the term “surgery” applies to surgical methods undertaken for removal of cancerous tissue, including pelvic lymphadenectomy, radical prostatectomy, transurethral resection of the prostate (TURP), excision, dissection, and tumor biopsy/removal.

In a particular embodiment, the patient has not been subject to hormonal therapy.

An increase in the likelihood of positive clinical outcome corresponds to a decrease in the likelihood of cancer recurrence.

In a third aspect, the present invention relates to an in vitro method for predicting the recurrence of prostate cancer in a patient, said method comprising the following steps of:
i. measuring the expression levels of miR-101 and miR-145 in a biological sample obtained from said subject;
ii. comparing said expression levels of miR-101 and miR-145 with their respective predetermined reference values; and
iii. predicting the recurrence of prostate cancer, wherein an expression level of miR-101 lower than the respective predetermined reference value and an expression level of miR-145 higher than the respective predetermined reference value is indicative that the patient has an increased likelihood of a recurrence of prostate cancer.

The term “recurrence” is used herein to refer to local or distant recurrence (i.e., metastasis) of cancer. For example, prostate cancer can recur locally in the tissue next to the prostate or in the seminal vesicles. The cancer may also affect the surrounding lymph nodes in the pelvis or lymph nodes outside this area. Prostate cancer can also spread to tissues next to the prostate, such as pelvic muscles, bones, or other organs.

In a particular embodiment, the method further comprises a step of measuring the expression level of miR-141.

Thus, the method comprises the following steps of:
i. measuring the expression levels of miR-101, miR-145 and miR-141 in a biological sample obtained from said subject;
ii. comparing said expression levels of miR-101, miR-145 and miR-141 with their respective predetermined reference values; and
iii. predicting the recurrence of prostate cancer, wherein an expression level of miR-101 lower than the respective predetermined reference value, an expression level of miR-145 higher than the respective predetermined reference value and an expression level of miR-141 higher than the predetermined reference value is indicative that the patient has an increased likelihood of a recurrence of prostate cancer.

In another particular embodiment, the method further comprises a step of measuring the expression level of miR195.

Thus, the method comprises the following steps of:
i. measuring the expression levels of miR-101, miR-145 and miR-195 in a biological sample obtained from said subject;
ii. comparing said expression levels of miR-101, miR-145 and miR-195 with their respective predetermined reference values; and
iii. predicting the recurrence of prostate cancer, wherein an expression level of miR-101 lower than the predetermined reference value, an expression level of miR-145 higher than the predetermined reference value and an expression level of miR-195 higher than the predetermined reference value is indicative that the patient has an increased likelihood of a recurrence of prostate cancer.

In a preferred embodiment, the method further comprises a step of measuring the expression levels of miR141 and miR195.

Thus, the method comprises the following steps of:
i. measuring the expression levels of miR-101, miR-145, miR-141 and miR-195 in a biological sample obtained from said subject;
ii. comparing said expression levels of miR-101, miR-145, miR-141 and miR-195 with their respective predetermined reference values; and
iii. predicting the recurrence of prostate cancer, wherein an expression level of miR-101 lower than the predetermined reference value, an expression level of miR-145 higher than the predetermined reference value, an expression level of miR-141 higher than the predetermined reference value and an expression level of miR-195 higher than the predetermined reference value is indicative that the patient has an increased likelihood of a recurrence of prostate cancer.

In another particular embodiment, the method further comprises a step of measuring the expression level of miR-375.

Thus, the method comprises the following steps of:
i. measuring the expression levels of miR-101, miR-145 and miR-375 in a biological sample obtained from said subject;
ii. comparing said expression levels of miR-101, miR-145 and miR-375 with their respective predetermined reference values; and
iii. predicting the recurrence of prostate cancer, wherein an expression level of miR-101 lower than the predetermined reference value, an expression level of miR-145 higher than the predetermined reference value and an expression level of miR-375 higher than the predetermined reference value is indicative that the patient has a increased likelihood of a recurrence of prostate cancer.

In a preferred embodiment, the method further comprises a step of measuring the expression levels of miR-375 and miR-141.

Thus, the method comprises the following steps of:
i. measuring the expression levels of miR-101, miR-145, miR-375 and miR-141 in a biological sample obtained from said subject;
ii. comparing said expression levels of miR-101, miR-145, miR-375 and miR-141 with their respective predetermined reference values; and
iii. predicting the recurrence of prostate cancer, wherein an expression level of miR-101 lower than the respective predetermined reference value, an expression level of miR-145 higher than the respective predetermined reference value, an expression level of miR-375 higher than the predetermined reference value and an expression level of miR-141 higher than the predetermined reference value is indicative that the patient has an increased likelihood of a recurrence of prostate cancer.

In another preferred embodiment, the method further comprises a step of measuring the expression levels of miR-375 and miR-195.

Thus, the method comprises the following steps of:
i. measuring the expression levels of miR-101, miR-145, miR-375 and miR-195 in a biological sample obtained from said subject;
ii. comparing said expression levels of miR-101, miR-145, miR-375 and miR-195 with their respective predetermined reference values; and
iii. predicting the recurrence of prostate cancer, wherein an expression level of miR-101 lower than the respective predetermined reference value, an expression level of miR-145 higher than the respective predetermined reference value, an expression level of miR-375 higher than the predetermined reference value and an expression level of miR-195 higher than the predetermined reference value is indicative that the patient has an increased likelihood of a recurrence of prostate cancer.

In still another preferred embodiment, the method further comprises a step of measuring the expression levels of miR-375, miR-141 and miR-195.

Thus, the method comprises the following steps of:
i. measuring the expression levels of miR-101, miR-145, miR-375, miR-141 and miR-195 in a biological sample obtained from said subject;
ii. comparing said expression levels of miR-101, miR-145, miR-375, miR-141 and miR-195 with their respective predetermined reference values; and
iii. predicting the recurrence of prostate cancer, wherein an expression level of miR-101 lower than the respective predetermined reference value, an expression level of miR-145 higher than the respective predetermined reference value, an expression level of miR-375 higher than the predetermined reference value, an expression level of miR-141 higher than the predetermined reference value and an expression level of miR-195 higher than the predetermined reference value is indicative that the patient has an increased likelihood of a recurrence of prostate cancer.

Measuring the expression level of miRs in a biological sample obtained from the patient may be performed by a variety of techniques as previously described.

In one embodiment, the patient is newly diagnosed with a clinically localized prostate cancer prior to or after primary therapy for clinically localized prostate cancer.

In a particular embodiment, the patient is diagnosed with a prostate cancer with a Gleason score of =3+4 or =4+3.

In another particular embodiment, the primary therapy for is surgery, radiation therapy including brachytherapy and external beam radiation therapy, high-intensity focused ultrasound (HIFU), chemotherapy, cryosurgery, hormonal therapy, or combination thereof.

The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention.

### Methods for Adjusting Treatments

In another aspect, the invention further provides methods for developing personalized treatment plans. Information gained by way of the methods described above can be used to develop a personalized treatment plan for a transplant recipient.

Accordingly, in a further aspect, the present invention relates to a method for adjusting the treatment administered to a patient diagnosed with a clinically localized prostate cancer, comprising the following steps of (i) performing predictive methods of the present invention n, and (ii) adjusting the treatment.

As used herein, the term “adjusting” refers to modulating, changing or adapting the treatment administered to a patient when the patient is diagnosed with the methods as described above.

The treatment may consists of surgery, radiation therapy including brachytherapy and external beam radiation therapy, high-intensity focused ultrasound (HIFU), chemotherapy, cryosurgery, hormonal therapy, or combination thereof.

### Kits of the Invention

The invention also relates to a kit suitable for performing the methods of the invention wherein said kit comprises means for measuring the expression level of at least miR-101 and miR-145 and additionally the expression level of miR-141 and/or miR-195 and/or miR-375 in a biological sample obtained from the subject. The kits may include probes, primers, macroarrays or microarrays as described above. For example, the kit may comprise a set of miRNA probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards.

Alternatively the kits of the invention may comprise amplification primers (e.g. stem-loop primers) that may be pre-labelled or may contain an affinity purification or attachment moiety. The kit may further comprise amplification reagents and also other suitably packaged reagents and materials needed for the particular amplification protocol.

The invention will be further illustrated by the following figures and examples. However, these examples and figures should not be interpreted in any way as limiting the scope of the present invention.

### FIGURES

FIG. 1: ROC analysis for circulating miR-101+miR-145. ROC analysis for the discrimination of circulating miRNA levels in patients without or with prostate cancer (sensitivity=92.1%; specificity=95.6%, AUC=98.1%, p<0.01)

FIG. 2: ROC analysis for circulating miR-375+miR-141+miR-145+miR-101. ROC analysis for the discrimination of circulating miRNA levels in patients without or with prostate cancer (sensitivity=92.1%; specificity=95.5%, AUC=98.6%, p<0.01)

### MATERIAL & METHODS

Participants and Recruitment

This cohort prospective study was conducted over three years in University-affiled hospitals (Inserm Unit 1065, Nice and department of Urologie, Hospital of Nice).

For the prostate cancer patient population (n=40), the inclusion criteria were: patients between 45 to 75 year-old, treated by radical prostatectomy in the department of Urology at Nice hospital for localized or localized advanced prostate cancer. The clinico-biologic parameters for the patients were indicated in Table 1.

For the control patient group (n=25), the criteria of inclusion were age below 45 years, without prostatic background or chronic disease that might modify microRNA expression profile. This study was conducted according to the Declaration of Helsinki for Medical Research involving Human Subjects and the Ethics Committee. The Medical Review Board of the ART center approved the protocol. Informed consent was obtained for all patients.

TABLE 1
Clinico-biologic parameters for the prostate cancer patients:

| Patients variables | | |
|---|---|---|
| Age mediane [min-max] (n) | 65.5 [50-72] (40) | |
| PSA (ng/ml) ± SD | 7.7 ± 2.33 | |
| Tumor stage: n (%) | | |
| pT1c | 1 | (2.5) |
| pT2a | 3 | (7.5) |
| pT2c | 23 | (57.5) |
| pT3a | 9 | (22.5) |
| PT3b | 4 | (10) |
| Gleason score n (%) | | |
| Gleason 6 | 2 | (5) |
| Gleason 7 | 36 | (90) |
| Gleason 8 | 2 | (5) |
| Gleason pattern n (%) | | |
| Pattern 3 | 32 | (80) |
| Pattern 4 | 8 | (20) |
| D'Amico score n (%) | | |
| Low Risk | 9 | (22.5) |
| Intermediate Risk | 23 | (57.5) |
| High Risk | 8 | (20) |
| CAPRA score | | |
| Low Risk | 14 | (35) |
| Intermediate Risk | 25 | (62.5) |
| High Risk | 1 | (2.5) |

PSA = Prostatic Specific antigen;
SD = Standard Deviation

Blood Samples

Blood samples (10 ml) were taken on EDTA tubes before prostatectomy and transported immediately to the laboratory. Then, the tubes were centrifuged at 3000 RPM for 10 min at room temperature. Plasma was collected in eppendorff tubes and stored at −80° C. until use.

Preparation of Total RNA

Total RNA from 300 μl of plasma was purified using the Trizol® reagent (500 μl Invitrogen, Cergy Pontoise, France). As an external standard, 25 fmol of cel-miR-39 (Qiagen, Courtaboeuf, France) was added. The aqueous phase was precipitated with 1.5 vol of ethanol 100% and 1 l of glycoblue nucleic acid carrier (Life technologies, Saint-Aubin, France). Total RNA was further purified on column (High pure miRNA isolation kit, Roche Diagnostics, Meylan, France). The quantity of RNA was evaluated with BioTek's synergy 2 alpha microplate reader (Bio Tek, Colmar, France).

Real-Time RT-PCR Analysis of miRNA Expression

RT-PCR reactions were performed using the stem-loop RT-PCR method, which is specific for mature miRNA (TaqMan miRNA assays; Applied Biosystems, Foster City, Calif.). Ten nanograms of total RNA were reverse transcribed in a 7.5 μl reaction using Multiscribe Reverse Transcriptase and a TaqMan miRNA RT primer (Applied Biosystems). The reaction mixture was incubated at 16° C. for 30 min, 42° C. for 30 min, 85° C. for 5 min, and finally held at 4° C. until subsequent analysis or stored at −20° C. Five microliters of the reverse transcribed product (2-fold dilution from RT-PCR) were assayed using TaqMan Universal PCR Master Mix, no AmpErase, and 1 μl of TaqMan miRNA (Applied Biosystems) PCR primers/probe mix in a 15 μl reaction mix. Real-time RT-PCR was performed on a StepOne apparatus (Applied Biosystems) using the following conditions: after 10 min at 95° C., 40 cycles were performed at 95° C. for 15 sec, and 60° C. for 1 min. The data were normalized to cel-miR-39 using the ΔCt method.

Statistical Analyses

Multivariate regression analyses were carried out to assess the strength of the association between the miRNA score and the endpoint of interest. A probability <0.05 was considered to be statistically significant.

Results

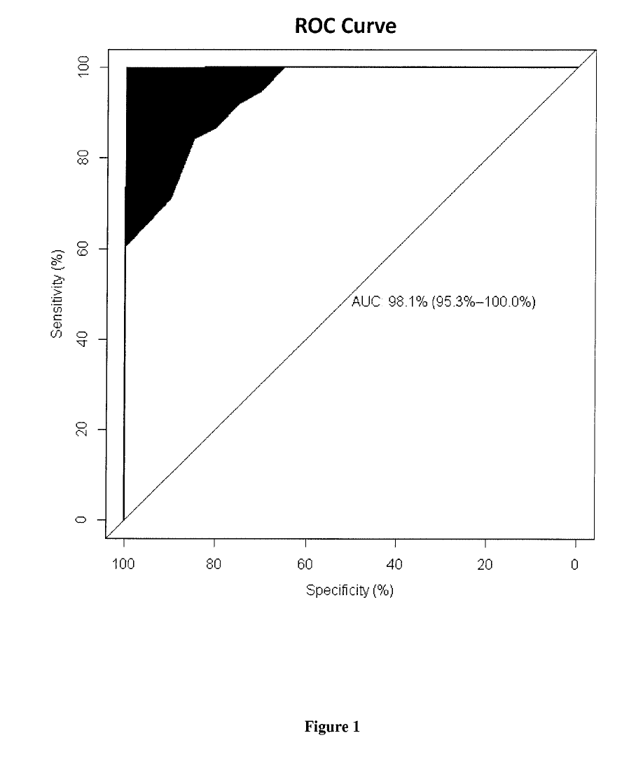

Multivariate analysis of the miR-101 and miR-145 levels showed a sensitivity of 92.1% and a specificity of 95.6% and an AUC of 98.1% (FIG. 1).

Multivariate analysis of the miR-101, miR-145 and miR-195 levels showed a sensitivity of 100% and a specificity of 95.5% and an AUC of 99.5%.

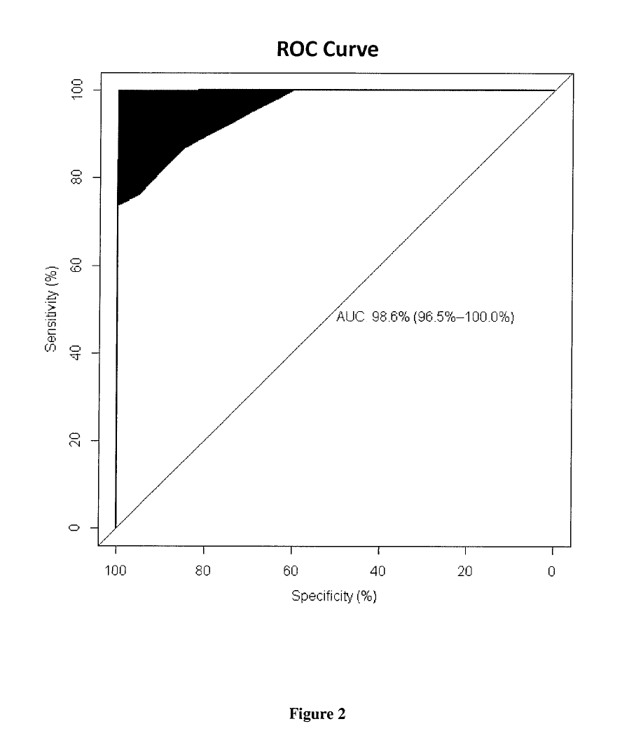

Multivariate analysis of the miR-375+miR-141+miR-145+miR-101 levels showed a sensitivity of 92.1% and a specificity of 95.5% and an AUC of 98.6% (FIG. 2).

### REFERENCES

Throughout this application, various references describe the state of the art to which this invention pertains. The disclosures of these references are hereby incorporated by reference into the present disclosure.
Sapre N, Selth L A; Circulating MicroRNAs as Biomarkers of Prostate Cancer: The State of Play; Prostate Cancer. 2013; 2013:539680.

1. An in vitro method for predicting or diagnosing a prostate cancer in a subject, said
method comprising the following steps of:
i. measuring the expression levels of miR-101 and miR-145 in a biological sample obtained from said subject;
ii. comparing said expression levels of miR-101 and miR-145 with their respective predetermined reference values; and
iii. determining whether the subject has or is at risk of having a prostate cancer, wherein an expression level of miR-101 lower than the predetermined reference value and an expression level of miR-145 higher than the predetermined reference value is indicative that the subject has, or is at risk of having a prostate cancer.

2. An in vitro method for predicting the clinical outcome for a patient diagnosed with prostate cancer, said method comprising the following steps of:
i. measuring the expression levels of miR-101 and miR-145 in a biological sample obtained from said subject;
ii. comparing said expression levels of miR-101 and miR-145 with their respective predetermined reference values; and
iii. predicting the clinical outcome for said patient, wherein an expression level of miR-101 lower than the respective predetermined reference value and an expression level of miR-145 higher than the respective predetermined reference value is indicative that the patient has a decreased likelihood of a positive clinical outcome.

3. An in vitro method for predicting the recurrence of prostate cancer in a patient, said method comprising the following steps of:
i. measuring the expression levels of miR-101 and miR-145 in a biological sample obtained from said subject;
ii. comparing said expression levels of miR-101 and miR-145 with their respective predetermined reference values; and
iii. predicting the recurrence of prostate cancer, wherein an expression level of miR-101 lower than the respective predetermined reference value and an expression 5 level of miR-145 higher than the respective predetermined reference value is indicative that the patient has an increased likelihood of a recurrence of prostate cancer.

4. The method according to claim 1, wherein the method further comprises a step of measuring the expression level of miR-141.

5. The method according to claim 1, wherein the method further comprises a step of measuring the expression level of miR-195.

6. The method according to claim 1, wherein the method further comprises a step of measuring the expression level of miR-375.

7. The method according to claim 1, wherein the biological sample is a blood sample or a urine sample.

8-12. (canceled)

13. The method of claim 2, wherein the method further comprises a step of measuring the expression level of one or more of miR-141, miR-195 and miR-375.

14. The method of claim 3, wherein the method further comprises a step of measuring the expression level of one or more of miR-141, miR-195 and miR-375.

15. The method of claim 2, wherein the biological sample is a blood sample or a urine sample.

16. The method of claim 3, wherein the biological sample is a blood sample or a urine sample.

17. The method of claim 1, further comprising a step of treating a subject diagnosed with prostate cancer with a method selected from the group consisting of: surgery, radiation therapy, high-intensity focused ultrasound (HIFU), chemotherapy, cryosurgery and hormonal therapy, or a combination thereof.

18. The method of claim 2, further comprising a step of treating a patient identified as having a decreased likelihood of a positive clinical outcome with a method selected from the group consisting of: surgery, radiation therapy, high-intensity focused ultrasound (HIFU), chemotherapy, cryosurgery and hormonal therapy, or a combination thereof.

19. The method of claim 3, further comprising a step of treating a patient identified as having an increased likelihood of a recurrence of prostate cancer with a method selected from the group consisting of: surgery, radiation therapy, high-intensity focused ultrasound (HIFU), chemotherapy, cryosurgery and hormonal therapy, or a combination thereof.
